(19) United States
(12) Patent Application Publication
(10) Pub. No.: US 2007/0014834 A1
(43) Pub. Date: Jan. 18, 2007

(54) Apparatus and methods of improved delivery of orally-administered therapeutic substances

(76) Inventors: Miles Lachlan McDowall, Eden Prairie, MN (US); Paul Haven McDowall, Eden Prairie, MN (US)

Correspondence Address: Paul H. McDowall, 12944 Cardinal Creek Road, Eden Prairie, MN, 55346, US

(21) Appl. No.: 11/404,664

(22) Filed: Apr. 14, 2006

Related U.S. Application Data
(60) Provisional application No. 60/671,856, filed on Apr. 15, 2005.

(51) Int. Cl. A61K 9/68; A23G 3/00

(52) U.S. Cl. 424/440; 424/776; 426/660

(57) ABSTRACT

The present invention pertains to flavor- and/or therapeutically-enhanced delivery of orally-administered medicines, natural substances, therapeutic materials and the like; in particular, the invention relates to apparatus and methods for rendering such items more attractive to a subject in need of same. In some embodiments a theobromine material and/or some marshmallow extract are included in combination with a whole or particles of, for example, a mentholated cough lozenge. The combination can be optionally coated with chocolate (and/or other) flavoring and/or coated, covered by or enveloped within a marshmallow or a marshmallow-flavored substance. The invention thus provides a family of consumable products that can be used to encourage patient compliance with a desired therapeutic regimen. Diverse single- and multiple-unit packaging is also included within the purview of the invention, including a kit of diverse materials including at least one of the inventive consumable products.

### CROSS REFERENCE TO RELATED APPLICATION

This patent disclosure claims the benefit of provisional U.S. patent application Ser. No. 60/671,856 filed 15 Apr. 2005 by Miles Lachlan McDowall et al. and entitled, “APPARATUS AND METHODS OF IMPROVED DELIVERY OF ORALLY-ADMINISTERED THERAPEUTIC SUBSTANCES,” (including all exhibits appended thereto) the contents of which are hereby incorporated herein in their entirety.

### FIELD OF THE INVENTION

The present invention pertains to flavor- and/or therapeutically-enhanced delivery of orally-administered medicines, natural substances, therapeutic materials and the like; in particular, the invention relates to apparatus and methods for rendering such items more attractive to a subject in need of same.

### BACKGROUND OF THE INVENTION

The present invention relates to improved formulations for confections which are intended to reside in the oral cavity for a period of time while being consumed. In particular, the prior art reveals a lack of enhanced delivery of diverse therapeutic substances.

Confections, especially medicinal tablets which deliver active ingredients in the oral cavity, are well known in the art and may be divided into various classes based upon their composition or intended effect. Examples include lozenges (so-called cough drops), compressed tablets and the like. The confections may have breath fresheners, breath deodorants, cough suppressants, nasal decongestants and the like.

Over the years, considerable effort has been directed to improving sensory perception in the oral cavity of the volatile oils contained in confections. Volatile oils or essential oils are derived from the leaves, stems or flowers of numerous plants and usually carry the savory or odorous principles of the plant which is obtained by distillation, expression or extraction. Volatile oils are known for their variable odors and distinctive tastes. The odors of volatile oils are modified by exposure to air, and the tastes of the volatile oils are quite distinctive. Some are sweet, while others can be mild, pungent, hot, acrid, caustic or burning in taste. In addition, some volatile oils can be made synthetically.

Enhancing the impact of the volatile oils in the oral cavity increases the benefit of the confection by ameliorating perceived bitterness, pungency, or other undesirable organoleptic sensations.

Menthol is isolated principally from the oil of Mentha arvensis. In its commercial form, menthol is present as crystals obtained from a process involving cooling of the above mentioned oil. Fractional distillation of peppermint oil which usually contains from about 50% to about 65% menthol provides another important source of menthol. In addition, menthol can be provided synthetically.

The use of menthol, for example, for its medicinal effect is known in the art. Menthol's cooling effect to the mouth is useful to relieve local irritations in the throat and mouth. Eucalyptus is another essential oil often combined with other essential oils such as menthol in confection formulations to impart medicinal effect. In particular, eucalyptus is believed to exhibit an expectorant action. The combination of the essential oils of menthol and eucalyptus, in a formulation capable of dissolving in the oral cavity provide a useful medicinal preparation in treatment of coughs and minor mouth, throat, and upper respiratory irritations.

Confections, which include such medicinal formulations, e.g., cough drops, lozenges, etc., however, suffer from several shortcomings.

For example, bitterness is often perceived due to the high potency of the essential oils which contain menthol. The bitterness of the menthol released in the oral cavity, therefore, provides an unpleasant organoleptic experience to the user thus reducing the likelihood of continued treatment with the lozenge or tablet. In addition, prior art preparations containing menthol suffer from reduced efficacy due to the erratic release of the menthol contained within the confection. Consequently, the cooling effect of menthol has often been attenuated. These problems, therefore, tend to detract from the acceptance of menthol-containing products as adjuncts in cough and cold therapy.

Other confectionery products, which may contain menthol or other flavorants derived from essential oils, such as spearmint, and/or peppermint, have also been known to produce bitterness while residing in the oral cavity.

While not an essential oil, the fruits of various species of capsicum are often added to food preparations to impart a pungent taste. Capsicum generally refers to various types of pepper of varying degrees of pungency. Capsicum oleoresin is an extract of fruits from various capsicum species and consists of a resinous matter and a liquid phase. The capsicum oleoresin is extremely pungent. For example, a dilution of one part of capsicum oleoresin in five million parts of 9% sugar water at 10.degree. C. produces a distinct burning effect in the throat and posterior region of the oral cavity. The capsicum oleoresin, with its characteristic peppery odor and extremely high bite, provides a useful source of aromas and is useful as an additive in various condiments, sauces and other foods. The traditional use of capsicum, therefore, is to provide a peppery heat sensation or spicy bite to foods or other confectionery items.

In the past, capsicum has generally been limited to those uses which rely on its peppery hot sensation or bite properties. U.S. Pat. No. 4,198,393 to Yoshida et al., discloses using cyclic acetals of 2-methyl-2-pentenal with capsicum and volatile oils, to provide flavor compounds that demonstrate a primary flavor taste and a purported etherial effect.

U.S. Pat. No. 4,420,472 to Boden, et al. discloses the use of prenyl methyl carbonate to enhance the flavor and aroma of chewing gums, toothpaste and medicinal products containing various flavor adjuvants including capsicum and other black pepper oleoresins as well as numerous volatile oils.

U.S. Pat. No. 4,197,328 to Sprecker, et al. discloses the use of oxabicyclooctanes to augment or enhance a variety of flavors and fragrances in various consumable materials. Similar to Yoshida, et al., capsicum and various volatile oils are among possible co-ingredients used as a flavor adjuvants enhanced by the oxabicyclooctanes.

U.S. Pat. No. 4,423,030 to Hayes, et al. discloses dental creams or mouthwashes having “two-tone flavors” provided by an essential oil component comprising about 0.01-5% by weight and a water-insoluble oleoresin component comprising about 0.001-0.1% by weight. Capsicum varieties are included among the oleoresins and are relied upon to provide a sensation of pungency different from that provided by the essential oil.

One of the components of chocolate, theobromine is known to inhibit sensory nerve activation and cough, among other effects. As reported in late 2004 by Omar S. Usmani, Maria G. Belvisi, Hema J. Patel, Natascia Crispino, Mark A. Birrell, Ma rta Korbonits, Dezso Korbonits, and Peter J. Barnes of the Imperial College (United Kingdom). Excerpts of the report follows. Cough is a common and protective reflex, but persistent coughing is debilitating and impairs quality of life. Antitussive treatment using opioids is limited by unacceptable side effects, and there is a great need for more effective remedies. The present study demonstrates that theobromine, a methylxanthine derivative present in cocoa, effectively inhibits citric acid-induced cough in guinea-pigs in vivo. Furthermore, in a randomized, double-blind, placebo-controlled study in man, theobromine suppresses capsaicin-induced cough with no adverse effects. We also demonstrate that theobromine directly inhibits capsaicin-induced sensory nerve depolarization of guinea-pig and human vagus nerve suggestive of an inhibitory effect on afferent nerve activation. These data indicate the actions of theobromine appear to be peripherally mediated. We conclude theobromine is a novel and promising treatment, which may form the basis for a new class of antitussive drugs. The foregoing was drawn from the report dated Monday 22 Nov. 2004 by the identified authors.

Thus, researchers have recently discovered that this ingredient present in chocolate could help stop persistent coughs. According to research published online in FASEB Journal the team have discovered that theobromine, a derivative found in cocoa, is nearly a third more effective in stopping persistent coughs when compared with codeine, currently considered the best cough medicine.

Professor Peter Barnes, from Imperial College London and Royal Brompton Hospital, and one of the authors of the report, commented: “Coughing is a medical condition which affects most people at some point in their lives, and yet no effective treatment exists. While persistent coughing is not necessarily harmful it can have a major impact on quality of life, and this discovery could be a huge step forward in treating this problem.” The researchers from Imperial College London, Royal Brompton Hospital, St Bartholomew's Hospital and Chinoin Co. Ltd, Budapest, gave 10 healthy volunteers theobromine, a placebo or codeine at different times in a randomised double blind trial. To compare the effectiveness of each they measured the levels of capsaicin in the volunteers and compared these after giving the three options. Capsaicin is used in clinical research to cause coughing, and is used as an indicator to test the effectiveness of cough medicines. When the volunteers were given theobromine, the concentration of capsaicin required to produce a cough was around one third higher when compared with the group receiving a placebo. When the group received codeine they needed only marginally higher levels of capsaicin to produce coughing, compared with the placebo. Theobromine works by suppressing vagus nerve activity, which is responsible for causing coughing. The team also discovered that unlike standard cough treatments, theobromine caused no adverse effects on either the cardiovascular or central nervous systems. Professor Maria Belvisi, from Imperial College London and Royal Brompton Hospital, and one of the paper's authors, comments: “Not only did theobromine prove more effective than codeine, at the doses used it was found to have none of the side effects. Normally the effectiveness of any treatment is limited by the dosage you can give someone.

With theobromine having no demonstrated side effects in this study it may be possible to give far bigger doses, further increasing its effectiveness. “At the same time, theobromine may not have any of the side effects such as drowsiness. This means there will be no restrictions on when it can be taken. For example, people using heavy machinery or who are driving should not take codeine, but they could take theobromine.” Theobromine belongs to a class of alkaloid molecules known as methylxanthines. Methylxanthines naturally occur in as many as sixty different plant species and include caffeine (the primary methlyxanthine in coffee) and theophylline (the primary methylxanthine in tea). Theobromine is the primary methylxanthine found in products of the cocoa tree, theobroma cacao.

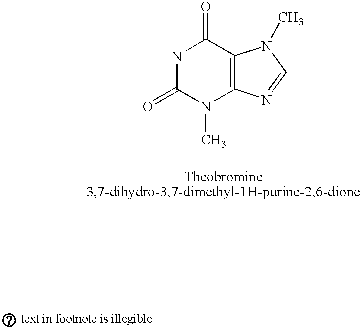

Theobromine affects humans similarly to caffeine, but on a much smaller scale. Theobromine is mildly diuretic (increases urine production), is a mild stimulant, and relaxes the smooth muscles of the bronchi in the lungs. In the human body, theobromine levels are halved between 6-10 hours after consumption. Theobromine has been used as a drug for its diuretic effect, particularly in cases where cardiac failure has resulted in an accumulation of body fluid. It has been administered with digitalis in order to relieve dilatation. Because of its ability to dilate blood vessels, theobromine also has been used to treat high blood pressure. Cocoa and chocolate products may be toxic or lethal to dogs and other domestic animals such as horses because these animals metabolize theobromine more slowly than humans. The heart, central nervous system, and kidneys are affected. Early signs of theobromine poisoning in dogs include nausea and vomiting, restlessness, diarrhea, muscle tremors, and increased urination or incontinence. The treatment at this stage is to induce vomiting. Cardiac arrhythmias and seizures are symptoms of more advanced poisoning. Different types of chocolate contain different amounts of theobromine. In general, theobromine levels are higher in dark chocolates (approximately 10 g/kg) than in milk chocolates (1-5 g/kg). Higher quality chocolate tends to contain more theobromine than lower quality chocolate. Cocoa beans naturally contain approximately 300-1200 mg/ounce theobromine

A need therefore exists in the art to improve delivery of orally-administered substances while providing a pleasurable experience with readily available materials.

### SUMMARY

The present invention pertains to flavor- and/or therapeutically-enhanced delivery of orally-administered medicines, natural substances, therapeutic materials and the like; in particular, the invention relates to apparatus and methods for rendering such items more attractive to a subject in need of same. In some embodiments a chocolate substance and/or active ingredients thereof (e.g., a methylxanthine such as theobromine) and/or some marshmallow extract (and/or marshmallow extract) are included in combination with one or more parts of a cough lozenge. In one form of this embodiment, a lozenge or particles thereof are optionally coated with a chocolate (and/or other) flavoring that in turn is covered by or enveloped within a marshmallow-flavored outer portion.

The invention thus provides a family of consumable products that can be used to encourage patient compliance with a desired therapeutic regimen. Diverse single- and multiple-unit packaging is also included within the purview of the invention, including a kit of diverse materials including at least one of the inventive consumable products. The foregoing and other aspects of the invention are provided with respect to a few select illustrated embodiments hereinbelow.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract of the invention is to enable the U.S. Patent and Trademark Office personnel and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

The foregoing and other aspects and features of the present invention will be more readily understood from the following detailed description of the embodiments thereof, when considered in conjunction with the drawings, in which like reference numerals indicate similar structures throughout the several views and the items illustrated are not drawn to scale. Those of skill in the art will readily recognize insubstantial changes, additions or modifications of the invention that are all expressly intended to be covered hereby.

### BRIEF DESCRIPTION OF THE DRAWINGS

FIG. 1 is a schematic illustration of the materials combined in one form of the invention to provide a final therapeutic product according to the basic teaching of the present invention.

FIG. 2 is a word-diagram describing some of the materials used to render a therapeutic product according to the invention.

FIG. 3 is yet another depiction of a way to combine materials to provide a therapeutic product according to the invention.

FIG. 4 is a perspective view illustrating another embodiment of the present invention wherein the therapeutic product comprises an elongated member.

FIG. 5 is a perspective view illustrating another embodiment of the present invention wherein the therapeutic product comprises a relatively short cylindrical member having a substantially centrally located portion of a material that is different than the outer material.

FIG. 6 depicts a multiple-product package for use in conjunction with, for example, a package box or a roll-type dispenser for a therapeutic product according to the invention.

FIG. 7 is a perspective view illustrating another embodiment of the present invention wherein the therapeutic product comprises a relatively short cylindrical member having substantially evenly distributed portions of a different materials.

FIG. 8 is a perspective view of another packaging regimen for a therapeutic product according to the invention.

FIG. 9 is an illustration representing a proposed commercially distributed kit containing at least one therapeutic product according to the invention as well as one or more related or desirable items that a subject might want to utilize in conjunction with the therapeutic product of the present invention.

### DETAILED DESCRIPTION OF THE ILLUSTRATED EMBODIMENTS

In the following detailed description, references are made to illustrative embodiments intended to assist the reader in appreciating some aspects of the present invention and are not intended as exhaustive examples of the full breadth or scope of the invention.

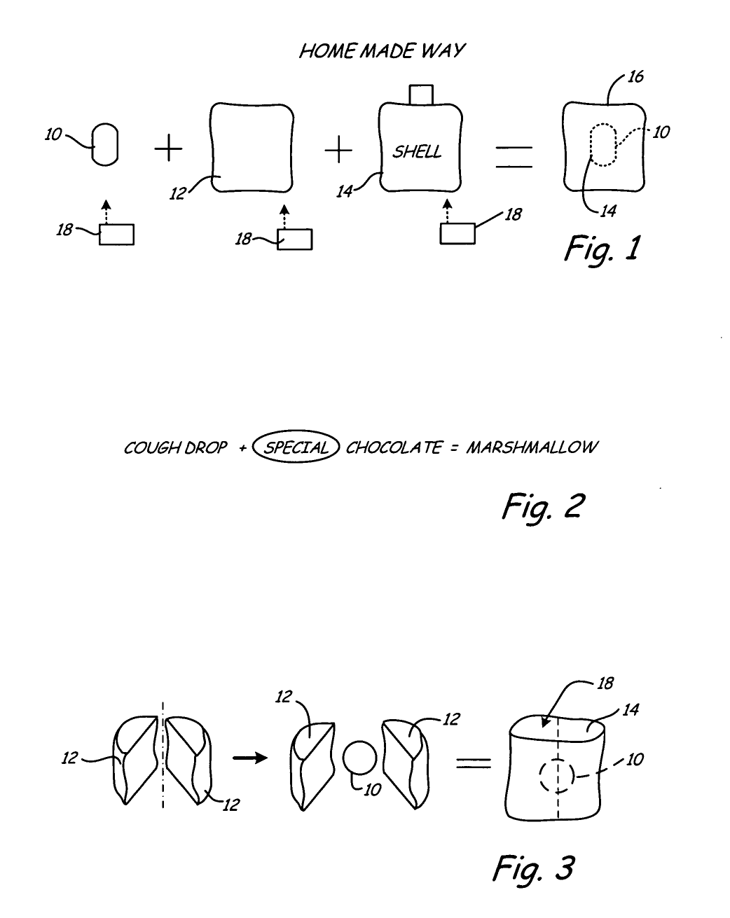

FIG. 1 is a schematic illustration captioned “home made way,” of some of the materials 10, 12, 14 that can be combined together in one form of the invention to provide a final therapeutic product 16 according to the basic teaching of the present invention. In one embodiment of the invention, the materials 10, 12, 14 include a relatively hard therapeutic member 10 (e.g., a cough drop), a relatively soft material 12 adapted to receive the relatively hard therapeutic member, and a material configured for providing a shell 14 of relatively sweet material (e.g., chocolate, honey, jelly, jam, syrup, etc.) over the other materials 10, 12. Alternatively the material 14 can comprise a material that tastes different than at least one of the other materials 10, 12 utilized to produce the final therapeutic product 16. One or more of the materials 10, 12, 14 can include a base material and optionally further additional materials 18, such as a holistic or natural therapeutic substance such as marshmallow root or the like (e.g., a natural cough suppressant). The additional materials 18 are depicted graphically in FIG. 1 by a square with a dashed line and arrow pointed toward materials 10, 12, 14. In one embodiment of the invention the product 16 comprises a commercially available cough drop 10 covered in rapidly hardening chocolate syrup 14 and inserted or wrapped in an outer layer of marshmallow 12. In a related embodiment the chocolate syrup 14 and/or the marshmallow 12 includes an amount of marshmallow root as the additional materials 18.

In another embodiment of the invention, the exterior of the product 16 comprises the hardened material 10 (e.g., a cough droplet) and the interior includes the relatively soft material 12 (e.g., marshmallow) and the shell material 14. Like the just-described embodiments additional materials 18 can be added or included to any one or all of the materials 10, 12, 14.

FIG. 2 is a word-diagram describing the materials used to render a therapeutic product 16 according to one embodiment of the invention—which roughly transcribed states, “cough drop plus special chocolate plus marshmallow.” In the described embodiment the term “special” can be interpreted to refer to additional materials 18 and/or the preferred rapidly hardening characteristic of the shell material 14.

FIG. 3 is yet another depiction of a way to combine materials to provide a therapeutic product 16 according to the invention. As depicted FIG. 3 is intended to convey a method for fabricating the product 16 by first dividing a soft material 12 into at least two opposing pieces, deploying a relatively hard material 10 between the opposing pieces of soft material 12 and joining the opposing pieces to envelop the hard material 10 (depicted by arrows in FIG. 3). The soft material 12 is then sealed to retain the material 10 within the material 12. As depicted in FIG. 3, an additional material 18 can be used to reconnect the material 12 such as honey or chocolate (with or without the rapidly hardening characteristic). The material 12 can also be reconnected using heat or other means known in the art of cooking and manufacturing of diverse confectionary products, ingredients and components.

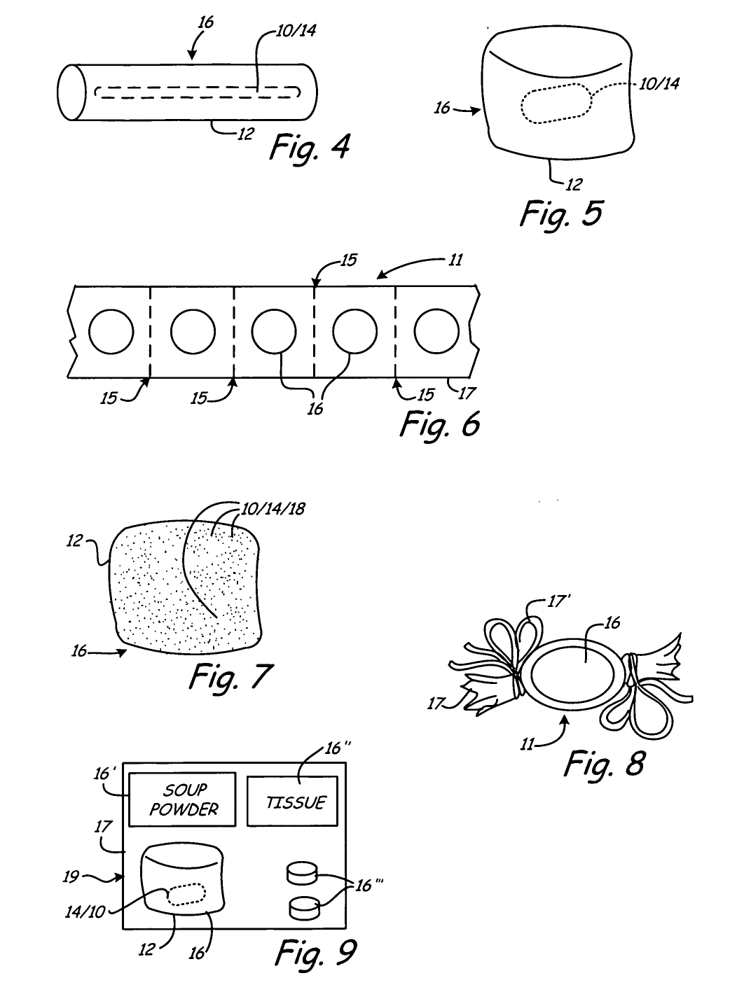

FIG. 4 is a perspective view illustrating another embodiment of the present invention wherein the therapeutic product 16 comprises an elongated member having materials 10, 14 distributed along an interior longitudinal axis of the material 12 of product 16. Of course, the materials 10, 14 can comprise discrete individual pieces distributed along an axis or randomly within the material 12. Also, as noted previously, one or more optional or additional materials 18 can be included in the product 16 or incorporated into one of the materials 10, 12, 14. While as depicted the materials 10, 14 appears fully embedded within material 12, one or more portions of the materials 10,14 can protrude through a surface portion of material 12, if desired.

FIG. 5 is a perspective view illustrating another embodiment of the present invention wherein the therapeutic product 16 comprises a relatively short cylindrical member having a substantially centrally located portion of a material 10, 14 that is different than the outer material 12. As with other embodiments of the invention, the product depicted in FIG. 5 can be manually produced or mass produced. One method of fabrication of the product 16 involves adding a fractional amount of material 12 (e.g., a viscous marshmallow-flavored material) into a cup or a plurality of cups and letting it harden a bit. Then the material 10 is deposited on a top surface of the material 12 in the cup. The cup can be shaped so that it serves as a retaining mold for a final product 16 of diverse shape. The shape of the cup can include any geometric configuration, irregular configuration and/or mimic the shape of a known article, animal, building, component or part, etc. The cup can include heating, cooling, rotational motion, vacuum and/or pressurizing elements or assemblies to assist the hardening, curing and/or processing of the materials 10, 12, 14 into a final product 16. Of course, an array of cups can be used to enhance production output and the cups so arrayed can be of diverse shape. Accordingly, in one manufacturing process the cups include diverse shapes and are adapted to receive the materials 10, 12, 14 and/or additional materials 18 from a plurality of dispensing valves. Furthermore, the materials can include diverse commercially available food coloring or ornamental shapes, facets, team logos, trademarks, words and the like on one or more major, minor, flat or curved surfaces of the product 16 to render it more visually pleasing or interesting to one or more members of a target customer audience or patient population. For example, one can imagine this aspect of the invention lending itself to mass market embodiments targeting persons who attend outdoor events and/or events wherein they might engage in loud cheering thereby possibly desiring or requiring that a therapeutic substance be applied to their throat, tongue, gums etc.

FIG. 6 depicts a multiple-product package 11 for use in conjunction with, for example, a package box or a roll-type dispenser (not depicted) for a therapeutic product 16 according to the invention. The product package 11 includes a substrate 17 including pocket-like features adapted to each receive at least one unit of product 16. The substrate 17 can comprise two layers of material that are adhered together using, for example, pressure-sensitive adhesive or heat-curable material. One of more sheets comprising the substrate 17 can be formed out of paper, resin-based or plastic materials that is rendered opaque and/or colored and can include words, logos, trademarks and the like printed or formed thereon. The substrate 17 can include perforations 15 disposed between units of the product 16 to ease manual dispensing of a desired number of units of the product 16.

FIG. 7 is a perspective view illustrating another embodiment of the present invention wherein the therapeutic product 16 comprises a relatively short cylindrical member having substantially evenly distributed portions of a different materials 10, 12, 14 (and optionally, additional materials 18). Of course the product 16 can be configured into diverse shapes as previously described.

FIG. 8 is a perspective view of another packaging regimen 11 for a therapeutic product 16 according to the invention wherein the product is wrapped in an outer layer, or substrate, with material 17 and the a pair of elongated ribbon or string-like members 17′ journal around opposing sides of the product 16. The materials can be colored for any occasion including metallic-looking ribbons 17′ or the like as desired for any constituent patient, targeted customer or to match the decor of a physical or geographic location.

FIG. 9 is an illustration representing an embodiment for a commercially distributed kit 19 containing at least one therapeutic product 16 according to the invention as well as one or more related or desirable items 16′,16″,16″′ that a subject or patient might want to utilize in conjunction with the therapeutic product 16 of the present invention. The kit 19 includes a substrate 17 adapted to constrain the product 16 and at least one of the other related or desirable items 16′, 16″, 16′″. As depicted the kit 19 includes at least one unit of product 16 and, for example, a portion of powdered soup 16′, some facial tissue 16″, and two aspirin 16′″. Of course, more, less or other items can be included in the kit 19 and all such instantiations of the kit 19 are intended to be covered by the claimed invention as long as said kit 19 includes at least one product 16. One or more kit 19 can be packaged for sale as an over-the-counter cough or cold remedy or via an untended (vending) machine. In addition to product 16, an over-the-counter medicine or substance can be included with the kit 19, such as an antacid, inhaler and the like.

It should be understood that, certain of the above-described structures, functions and operations of the illustrated embodiments are not necessary to practice the present invention and are included in the description simply for completeness of a exemplary embodiment or embodiments. It will also be understood that there may be other materials, structures, processes, functions and operations ancillary to the described and depicted embodiments of the invention that are not expressly disclosed and are not necessary to the practice of the present invention. For example, although the invention has been described and depicted as a discrete article fit for immediate human consumption the product can be rendered into liquid form during initial fabrication (e.g., a cough syrup-like preparation) or can be blended or melted by a patient. If blended the product can be combined with other materials such as ice cream, fruit juice or fruit and literally blended together to create a so-called smoothie. If melted or otherwise the product can be placed on a cake, placed on a stick, frozen or re-frozen and the like.

In addition, it will be understood that specifically described structures, functions and operations set forth hereinabove can be practiced in conjunction with the present invention, but they are also not essential to its practice. It is therefore to be understood, that within the scope of the appended claims, the invention may be practiced otherwise than as specifically described without actually departing from the spirit and scope of the claimed invention.

1. A method of fabricating a consumable product, comprising:
combining a volume of chocolate with at least one of: a medicinal tablet member, a cough lozenge member, a pharmaceutical-containing pill member; and
adding at least one of a marshmallow confection, a marshmallow root extract, and a methylxanthine to the combined member and chocolate.

2. A method according to claim 1, wherein the methylxantine comprises theobromine.

3. A method according to claim 1, further comprising, prior to performing the combining step: converting the member into discrete portions of reduced size.

4. A method according to claim 1, wherein the volume of chocolate comprises one of a powdered volume of chocolate and a melted volume of chocolate.

5. A method according to claim 4, wherein the combining step comprises coating the member with the melted volume of chocolate.

6. A method according to claim 5, wherein the adding step comprises inserting the combined member and the chocolate into a marshmallow confection.

7. A method according to claim 1, wherein the adding step comprises inserting the combined member and the chocolate between two parts of a marshmallow confection, and further comprising sealing the two parts of the marshmallow confection together.

8. A method according to claim 1, wherein the steps of combining and adding are performed as a two step co-extrusion process.

9. A method according to claim 8, further comprising dicing the extruded material into individual units.

10. A method according to claim 9, further comprising depositing each one of a plurality of the consumable product into a sterile package.

11. A method according to claim 10, wherein said sterile package comprises a plurality of connected sterile packages.

12. A method according to claim 11, further comprising a plurality of perforations disposed between adjacent ones of said plurality of connected sterile packages.

13. A consumable article, comprising:
a volume of chocolate combined with at least one of: a medicinal tablet member, a cough lozenge member, a pharmaceutical-containing pill member; and
at least one of a marshmallow confection, a marshmallow root extract, and a methylxanthine.

14. A consumable article according to claim 13, wherein the volume of chocolate and the member are inserted within the marshmallow confection.

15. A consumable article according to claim 13, wherein the volume of chocolate comprises one of a powered volume of chocolate and a solid volume of chocolate.

16. A consumable article according to claim 13, wherein the methylxanthine comprises theobromine.

17. A kit, comprising:
a consumable article, said consumable article further comprising:
a volume of chocolate combined with at least one of: a medicinal tablet member, a cough lozenge member, a pharmaceutical-containing pill member; and
at least one of a marshmallow confection, a marshmallow root extract, and a methylxanthine; and
at least one other consumable product.

18. A kit according to claim 17, wherein the at least one other consumable product comprises: an aspirin, an anti-inflammatory substance, a pain reducing preparation, a portion of powdered soup, at least one facial tissue, a cough lozenge, an amount of an essential oil.

19. A kit according to claim 17, further comprising a tamper-indicating seal coupled to said kit.

20. A kit according to claim 17, wherein the consumable article is configured as one of a cylindrical shape, a substantially planar shape, an elongated shape, an ovoid shape.
